(19) United States
(12) Patent Application Publication
(10) Pub. No.: US 2024/0093291 A1
(43) Pub. Date: Mar. 21, 2024

(54) SPATIAL GENOMICS WITH SINGLE CELL RESOLUTION

(71) Applicant: THE REGENTS OF THE UNIVERSITY OF CALIFORNIA, Oakland, CA (US)

(72) Inventors: Cyrille L. Delley, Oakland, CA (US); Adam Abate, Oakland, CA (US)

(73) Assignee: CZ BIOHUB SF, LLC, San Francisco, CA (US)

(21) Appl. No.: 18/266,085

(22) PCT Filed: Dec. 13, 2021
(86) PCT No.: PCT/US21/63114

§ 371 Date: Jun. 8, 2023

Related U.S. Application Data
(60) Provisional application No. 63/125,128, filed on Dec. 14, 2020.

(51) Int. Cl. C12Q 1/6874

(52) U.S. Cl. CPC C12Q 1/6874

(57) ABSTRACT

The present disclosure provides materials and methods for sequencing a tissue sample and that allows spatial information about the tissue to be recovered by sequencing approaches at a single cell level.

### STATEMENT REGARDING FEDERALLY SPONSORED RESEARCH

This invention was made with government support under grant no. U01 AI129206 awarded by The National Institutes of Health. The government has certain rights in the invention.

### FIELD

The present disclosure relates generally to methods for sequencing a tissue sample and obtaining spatial information about the tissue to be recovered by sequencing approaches at a single cell level.

### BACKGROUND

Single-cell sequencing has demonstrated unappreciated cellular diversity in many ostensibly homogeneous systems over the past few years and led to an ongoing scientific revolution in cell biology (Klein, A. M., et al., (2015) Cell, 161, 1187-1201; Macosko, E. Z., et al. (2015) Cell, 161, 1202-1214; Zheng, G. X. Y., et al. (2016) Nat. Commun., 8, 065912; Pellegrino, M., et al. (2018) Genome Res., 28, 1345-1352). Droplet microfluidics and deep sequencing are the major drivers behind this revolution (Klein et al., supra). However, droplet microfluidics requires a cell suspension as input material and thus all spatial information, such as the relative position of different cells to each other and the subcellular location of biomolecules, is lost.

Using advanced microscopy, it is possible to detect individual transcripts with hybridization methods like fluorescence in situ hybridization (FISH) and its derivatives, or to perform in situ sequencing (Langer-Safer, P. R., et al. (1982) Proc. Natl. Acad. Sci. U.S.A, 79, 4381-5; Raj, A., et al., (2008) Nat. Methods, 5,877-879; Lee, J. H., et al. (2014) Science, 343, 1360-1363). Although these orthogonal methods retain very high spatial resolution, including subcellular location, FISH methods cannot profile the entire transcriptome and in situ sequencing has low detection efficiency, and both are only available to expert laboratories. Recently, Slide-seq was introduced as a more user-friendly approach (Stahl, P. L., et al. (2016) Science, 353, 78-82; Rodriques, S. G., et al., (2019) Science, 363, 1463-1467). Slide-seq uses an array of barcoded oligos placed in known spatial position at resolutions of 10-100 μm. This barcode array is then overlaid with a tissue section. Permeabilization of the cells within the section enables the RNA transcripts to diffuse out and hybridize with the barcode oligos in close proximity on the array. After reverse transcription, the now barcoded transcripts can be amplified and converted into a sequencing library analogous to the libraries obtained from a droplet microfluidic single-cell experiment. However, in contrast to droplet single-cell sequencing where each barcode reveals a single-cell identity, here the barcodes reveal the spatial position on the array which was in close proximity to the cell that harbored the particular transcript. Over the whole library, this data set yields whole transcriptome information for each cell with spatial resolution on the order of 100 μm.

Since a typical mammalian cell is ˜10 μm, Slide-seq does not resolve single-cells but averages over tens to hundreds of cells in the vicinity of each array spot. Slide-seq thus provides a middle ground: starting from barcoded slides, which became recently commercially available, the experimental process is as easy as droplet workflows; it provides spatial information in contrast to droplets; but not at single-cell, like droplets or subcellular resolution as the microscopy approaches. Thus, there remains a need for a method of obtaining spatial information linked with sequence information at single-cell precision.

### SUMMARY OF THE INVENTION

One aspect of the present disclosure provides a method of determining the sequence and location of a nucleic acid in a tissue sample, said method comprising the steps of: (a) preparing a tissue sample, wherein said preparing comprises embedding the tissue sample in an embedding gel precursor solution and contacting the tissue sample with a lattice under conditions that allow gelation of the tissue sample and thereby forming a gelated tissue sample; (b) preparing a labeling substrate, wherein said labeling substrate comprises a first binding moiety comprising a spatial barcode molecule, wherein said first binding moiety is capable of binding to (i) a biomolecule in the gelated tissue sample; (ii) a biomolecule released from the gelated tissue sample; and/or (iii) a gel scaffold of the gelated tissue sample, and wherein said biomolecule or gel scaffold comprises a plurality of nucleic acid molecules; (c) incubating the labeling substrate of (b) with the gelated tissue sample of (a) under conditions that allow the first binding moiety to bind to the biomolecule or gel scaffold, thereby labeling the biomolecule or gel scaffold with a spatial barcode; (d) preparing a suspension solution comprising a labeled biomolecule or labeled gel scaffold from (c) and incubating said solution with a solution comprising a second binding moiety comprising a sequence barcode, under conditions that allow the second binding moiety to bind to a nucleic acid in the labeled biomolecule or labeled gel scaffold of (c), thereby further labeling the biomolecule or gel scaffold with the sequence barcode; and (e) determining the sequence and location of the nucleic acid.

In another embodiment, the present disclosure provides a method of determining the sequence and location of a nucleic acid in a tissue sample, said method comprising the steps of: (a) preparing a tissue sample, wherein said preparing comprises expanding the tissue sample with an expansion gel precursor solution and thereby forming an expanded tissue sample; (b) preparing a labeling substrate, wherein said labeling substrate comprises a first binding moiety comprising a spatial barcode molecule, wherein said first binding moiety is capable of binding to (i) a biomolecule in the expanded tissue sample; (ii) a biomolecule released from the expanded tissue sample; and/or (iii) a gel scaffold of the expanded tissue sample, and wherein said biomolecule or gel scaffold comprises a plurality of nucleic acid molecules; (c) incubating the labeling substrate of (b) with the expanded tissue sample of (a) under conditions that allow the first binding moiety to bind to the biomolecule or gel scaffold, thereby labeling the biomolecule or gel scaffold with a spatial barcode; (d) preparing a suspension solution comprising a labeled biomolecule or labeled gel scaffold from (c) and incubating said solution with a solution comprising a second binding moiety comprising a sequence barcode, under conditions that allow the second binding moiety to bind to a nucleic acid in the labeled biomolecule or labeled gel scaffold of (c), thereby further labeling the biomolecule or gel scaffold with the sequence barcode; and (e) determining the sequence and location of the nucleic acid.

In still another embodiment, the present disclosure provides a method of determining the sequence and location of a nucleic acid in a tissue sample, said method comprising the steps of: (a) preparing a tissue sample, wherein said preparing comprises (i) embedding the tissue sample in an embedding gel precursor solution and contacting the tissue sample with a lattice under conditions that allow gelation of the tissue sample and thereby forming a gelated tissue sample, and (ii) expanding the tissue sample to form a gelated, expanded tissue sample; (b) preparing a labeling substrate, wherein said labeling substrate comprises a first binding moiety comprising a spatial barcode molecule, wherein said first binding moiety is capable of binding to (i) a biomolecule in the gelated, expanded tissue sample; (ii) a biomolecule released from the gelated, expanded tissue sample; and/or (iii) a gel scaffold of the gelated, expanded tissue sample, and wherein said biomolecule or gel scaffold comprises a plurality of nucleic acid molecules; (c) incubating the labeling substrate of (b) with the gelated, expanded tissue sample of (a) under conditions that allow the first binding moiety to bind to the biomolecule or gel scaffold, thereby labeling the biomolecule or gel scaffold with a spatial barcode; (d) preparing a suspension solution comprising a labeled biomolecule or labeled gel scaffold from (c) and incubating said solution with a solution comprising a second binding moiety comprising a sequence barcode, under conditions that allow the second binding moiety to bind to a nucleic acid in the labeled biomolecule or labeled gel scaffold of (c), thereby further labeling the biomolecule or gel scaffold with the sequence barcode; and (e) determining the sequence and location of the nucleic acid.

In yet another embodiment, the present disclosure provides an aforementioned method wherein the biomolecule is selected from the group consisting of a nucleic acid, a lipid, a small molecule, a sugar, a protein, a nuclei, and a cell. In another embodiment, the tissue sample is about of 0.1 to 1,000 micron thickness. In one embodiment, the tissue sample is a human or animal biopsy. In still another embodiment, the biopsy comprises a sample from a tumor, lymphatic tissue, infected tissue, immune infiltrated tissue, central nervous system tissue, digestive system tissue, developing tissue; whole animal section, and microbial community.

In another embodiment, the present disclosure provides an aforementioned method wherein the tissue sample is subjected to freezing and/or slicing. In other embodiments, preparing the tissue sample optionally comprises contacting the tissue sample or a portion of the tissue sample with a detection agent selected from the group consisting of an antibody, a nucleic acid, a protein, a dye, a bead. In some embodiments, the tissue sample, prior to preparing, is subjected to a treatment selected from the group consisting of imaging, spectroscopy, mass spectroscopy, enzymatic assays and FISH. In one embodiment, the detection agent optionally comprises a spatial barcode.

In another embodiment, the present disclosure provides an aforementioned method wherein step (a) occurs after step (b).

In still another embodiment, the present disclosure provides an aforementioned method wherein the substrate is a slide, an array, a hydrogel, a tissue, a suspension of particles, or a suspension of droplets. In other embodiment, the substrate comprises from about 1 to about 4×10¹² first binding moieties. In one embodiment, the first binding moieties are releasable from the substrate in designated locations. In another embodiment, the first binding moieties are fixed to the substrate in designated locations.

The present disclosure provides an aforementioned method wherein the first binding moiety is a moiety selected from the group consisting of a nucleic acid, a protein, a peptide, an antibody-DNA conjugate, an isotopes, a MS-detectable molecule, a DNA-conjugate, a peptide analog, and a nucleic acid analog, according to some embodiments. In other embodiments, the spatial barcode molecule is an oligonucleotide. In other embodiments, the incubating comprises direct contact between the tissue sample and the labeling substrate. In still other embodiments, the incubating comprises placing the tissue sample in close proximity to the labeling substrate.

In some embodiments, an aforementioned method is provided wherein the conditions that allow the first binding moiety to bind to a biomolecule comprises treating the tissue sample with a digesting agent or a surfactant. In one embodiment a cell wall our cell outer membrane is digested. In another embodiment, the present disclosure provides an aforementioned method wherein the conditions that allow the first binding moiety to bind to a biomolecule comprises thawing the tissue sample.

In still another embodiment, the present disclosure provides an aforementioned method wherein 2, 3, 4, 5, 6, 7, 8, 9 or 10 or more labeling substrates are incubated with different sections of the tissue sample. In yet another embodiment, the present disclosure provides an aforementioned method wherein the labeled biomolecule of step (c), prior to preparing a suspension according to step (d), is subjected to a treatment selected from the group consisting of imaging, spectroscopy, mass spectroscopy, enzymatic assays and FISH.

In yet another embodiment, the present disclosure provides an aforementioned method wherein the preparing a suspension solution comprising the labeled biomolecule comprises digesting, grinding or slicing the tissue sample melting or hydrolysis of gels or tissue sample. In another embodiment, the present disclosure provides an aforementioned method wherein the second binding moiety comprising the sequence barcode is a bead. In one embodiment, the bead is a hydrogel bead.

In another embodiment, the present disclosure provides an aforementioned method wherein the second binding moiety comprises a unique sequence barcode associated with from about 1 to about 1×10⁶ primer sequences. In one embodiment, the sequence barcode includes a primer sequence for reverse transcription. In another embodiment, the present disclosure provides an aforementioned method wherein the nucleic acid is RNA. In one embodiment, the RNA is mRNA. In still another embodiment, the present disclosure provides an aforementioned method wherein a biomolecule or multiple biomolecules are captured in a hydrogel sample. In still another embodiment, the present disclosure provides an aforementioned method wherein the sequence and location of the nucleic acid is determined by sequencing and optionally one or more of MS, droplet assay, spectroscopy, and fluorescence.

In yet another embodiment, the present disclosure provides an aforementioned method wherein the embedding gel precursor solution comprises a photopolymerizable, dissolvable hydrogel precursor comprising, and wherein the lattice is a patterned photomask. In another embodiment, the present disclosure provides an aforementioned method wherein the incubating comprises direct contact between the tissue sample and the labeling substrate, and wherein the tissue sample is cross-linked to the labeling substrate under conditions that allow fractions of the tissue sample to be captured in hydrogel samples

In one embodiment, the present disclosure provides a method of determining the sequence and location of a nucleic acid in a tissue sample, said method comprising the steps of: (a) preparing a tissue sample of about 0.1 to 1,000 micron thickness, wherein said preparing comprises embedding the tissue sample in an embedding gel precursor solution and contacting the tissue sample with a lattice under conditions that allow gelation of the tissue sample, wherein the embedding gel precursor solution comprises a photopolymerizable and/or photodissolvable hydrogel precursor, and wherein the lattice is a patterned photomask; (b) preparing a labeling substrate, wherein said labeling substrate comprises a first binding moiety comprising an oligonucleotide spatial barcode molecule, wherein said first binding moiety is capable of binding to a gel scaffold, cell or nuclei in a gelated tissue sample of (a), wherein said gel scaffold, cell or nuclei comprises a plurality of nucleic acid molecules, wherein the first binding moiety is a nucleic acid and is releasable from the labeling substrate in pre-determined, designated locations; (c) incubating the labeling substrate of (b) with the gelated tissue sample of (a) under conditions that allow the first binding moiety to bind to the gel scaffold, cell or nuclei and thereby labeling the gel scaffold with a spatial barcode, cell or nuclei with the spatial barcode, wherein the incubating comprises direct contact between the tissue sample and the labeling substrate under conditions that allow diffusion of the first binding moiety between the gelated tissue sample and the labeling substrate; (d) preparing a suspension solution comprising the labeled gel scaffold, cell or nuclei from (c) and incubating said solution with a solution comprising a second binding moiety comprising an oligonucleotide sequence barcode under conditions that allow the second binding moiety to bind to a nucleic acid in the labeled gel scaffold, cell or nuclei, thereby further labeling the gel scaffold, cell or nuclei with the oligonucleotide sequence barcode, wherein the second binding moiety is a bead comprising between 100-1,000 primer sequences; and (e) determining the sequence and location of the nucleic acid.

In still another embodiment, a method of determining the sequence and location of a nucleic acid in a tissue sample is provided, said method comprising the steps of: (a) preparing a tissue sample of about 0.1 to 1,000 micron thickness, wherein said preparing comprises expanding the tissue sample with an expansion gel precursor solution, wherein the expansion gel precursor solution comprises acrylamide; (b) preparing a labeling substrate, wherein said labeling substrate comprises a first binding moiety comprising an oligonucleotide spatial barcode molecule, wherein said first binding moiety is capable of binding to a gel scaffold, cell or nuclei in an expanded tissue sample of (a), wherein said gel scaffold, cell or nuclei comprises a plurality of nucleic acid molecules, wherein the first binding moiety is a nucleic acid and is releasable from the labeling substrate in pre-determined, designated locations; (c) incubating the labeling substrate of (b) with the expanded tissue sample of (a under conditions that allow the first binding moiety to bind to the gel scaffold, cell or nuclei and thereby labeling the gel scaffold, cell or nuclei with the spatial barcode, wherein the incubating comprises direct contact between the tissue sample and the labeling substrate under conditions that allow diffusion of the first binding moiety between the expanded tissue sample and the labeling substrate; (d) preparing a suspension solution comprising the labeled gel scaffold, cell or nuclei from (c) and incubating said solution with a solution comprising a second binding moiety comprising an oligonucleotide sequence barcode under conditions that allow the second binding moiety to bind to a nucleic acid in the labeled gel scaffold, cell or nuclei, thereby further labeling the gel scaffold, cell or nuclei with the oligonucleotide sequence barcode, wherein the second binding moiety is a bead comprising between 100-1,000 primer sequences; and (e) determining the sequence and location of the nucleic acid.

In yet another embodiment, a method of determining the sequence and location of a nucleic acid in a tissue sample is provided, said method comprising the steps of: (a) preparing a tissue sample of about 0.1 to 1,000 micron thickness, wherein said preparing comprises (i) embedding the tissue sample in an embedding gel precursor solution and contacting the tissue sample with a lattice under conditions that allow gelation of the tissue sample, wherein the embedding gel precursor solution comprises a photopolymerizable and/or photodissolvable hydrogel precursor, and wherein the lattice is a patterned photomask, and (ii) expanding the tissue sample; (b) preparing a labeling substrate, wherein said labeling substrate comprises a first binding moiety comprising an oligonucleotide spatial barcode molecule, wherein said first binding moiety is capable of binding to a gel scaffold, cell or nuclei in a gelated tissue sample of (a), wherein said gel scaffold, cell or nuclei comprises a plurality of nucleic acid molecules, wherein the first binding moiety is a nucleic acid and is releasable from the labeling substrate in pre-determined, designated locations; (c) incubating the labeling substrate of (b) with the gelated tissue sample of (a) under conditions that allow the first binding moiety to bind to the gel scaffold, cell or nuclei and thereby labeling the gel scaffold, cell or nuclei with the spatial barcode, wherein the incubating comprises direct contact between the tissue sample and the labeling substrate under conditions that allow diffusion of the first binding moiety between the gelated tissue sample and the labeling substrate; (d) preparing a suspension solution comprising the labeled gel scaffold, cell or nuclei from (c) and incubating said solution with a solution comprising a second binding moiety comprising an oligonucleotide sequence barcode under conditions that allow the second binding moiety to bind to a nucleic acid in the labeled gel scaffold, cell or nuclei, thereby further labeling the gel scaffold, cell or nuclei with the oligonucleotide sequence barcode, wherein the second binding moiety is a bead comprising between 100-1,000 primer sequences; and (e) determining the sequence and location of the nucleic acid.

### BRIEF DESCRIPTION OF THE DRAWING

FIG. 1 shows one embodiment for an experimental setup of a label substrate with a cryosection of a tissue. FIG. 1A shows a dry hydrogel layer is spotted with “zipcode” oligos in known spatial positions. The hydrogel substrate is wetted with a mild cell lysis buffer and frozen. A frozen cryosection is placed over the Zipcode array and thawed to transfer the Zipcodes into the cells. Once thawed, the Zipcode DNA oligos diffuse into the tissue and label cells and cell nuclei in close proximity. These cells or nuclei can then be extracted and evaluated with any single-cell method (FIG. 1B). FIG. 1C provides an exemplary workflow with gellation.

FIG. 2 shows the labeling of tissue cryosections with DNA zipcodes. FIG. 2A) a possible encoding for a label substrate employing unique pairs of DNA barcodes. The spotting position of two DNA barcodes (A1 and H12) out of 96 are depicted. Each barcode pair is unique, hence A1 and H12 overlay at a single pixel. Barcode A1 and H12 have been mixed with fluorescein to provide a fiducial marks that can be visualized on a fluorescence microscope to cross validate tissue placement. FIG. 2B) Transmission image and FIG. 2C) fluorescence image of the same tissue view demonstrate successful transfer of FAM labeled DNA barcodes into the tissue, thus transferring a spatial encoding into the cells.

FIG. 3 shows a spatially resolved single-cell RNA experiment from the same mouse brain xenograft section displayed in FIG. 2A. FIG. 3A) Barcode rank plot in x versus total RNA or total ZIP-tag counts per barcode demonstrates that about 2,500 single cells have been processed. Right side, scatter plot of total RNA counts versus total ZIP-tag counts for each barcode. The true cell separate in 2D from the large background fraction. FIG. 3B) UMAP representation of the single-cell RNA profile from the cells identified in FIG. 3A. CLuster 1-4 correspond to mouse cells while cluster 0 and 5 are from the human xenograft. FIG. 3C) Reconstructed spatial organization of each cell colored according to cluster membership in FIG. 3B. For each cell the two most abundant associated spatial tags were used to assign the cell to a labelsubstrate grid position. Inserts show the transmission image of the same section (compare FIG. 2A), demonstrating that the spatial reconstruction correctly recovers the brain position and xenograft.

FIG. 4 shows the results of a spatial resolved single-cell chromosomal DNA genotyping experiment. FIG. 4A) Biopsy of a brain metastasis of a melanoma. FIG. 4B) Barcode rank plot for DNA amplicons and ZIP-tags. FIG. 4C) Heat map demonstrating reconstructed cell positions based on the ZIP-tags

### DETAILED DESCRIPTION

The present disclosure provides, in various embodiments described herein, methods to meet the aforementioned need in the art.

As described herein, in one embodiment of the present disclosure a method of determining the sequence and location of a nucleic acid in a tissue sample is provided comprising the steps of (a) preparing a tissue sample, wherein said preparing optionally comprises (i) embedding the tissue sample in an embedding gel precursor solution and contacting the tissue sample with a lattice under conditions that allow gelation of the tissue sample and thereby forming a gelated tissue sample, and/or (ii) expanding the tissue sample to form a gelated, expanded tissue sample; (b) preparing a labeling substrate, wherein said labeling substrate comprises a first binding moiety comprising a spatial barcode molecule, wherein said first binding moiety is capable of binding to (i) a biomolecule in the gelated, expanded tissue sample; (ii) a biomolecule released from the gelated, expanded tissue sample; and/or (iii) a gel scaffold of the gelated, expanded tissue sample, and wherein said biomolecule or gel scaffold comprises a plurality of nucleic acid molecules; (c) incubating the labeling substrate of (b) with the gelated, expanded tissue sample of (a) under conditions that allow the first binding moiety to bind to the biomolecule or gel scaffold, thereby labeling the biomolecule or gel scaffold with a spatial barcode; (d) preparing a suspension solution comprising a labeled biomolecule or labeled gel scaffold from (c) and incubating said solution with a solution comprising a second binding moiety comprising a sequence barcode, under conditions that allow the second binding moiety to bind to a nucleic acid in the labeled biomolecule or labeled gel scaffold of (c), thereby further labeling the biomolecule or gel scaffold with the sequence barcode; and (e) determining the sequence and location of the nucleic acid.

In some embodiments, the first binding moiety comprising the spatial barcode can bind anywhere within a particle or within the tissue sample (e.g., it can bind a cell, nuclei, gel scaffold, etc.) that will localize in the same compartment/droplet/particle as the target of interest (e.g., the nucleic acid that is sequences and spatially located within the sample) during application of the sequence barcode moiety. For example, the first binding moiety may bind to a nuclear envelope component (lipids, protein, etc.) or to a gel scaffold (e.g., a component of the gel matrix).

In one embodiment of the present disclosure, the feature size of mammalian cells in a tissue section are increased. By way of example, gel expansion, which has been described in the context of super resolution microscopy to isometrically expand the tissue section 4-16 fold (Chen, F., et al., (2015) Science, 347, 543-8; Chang, J.-B., et al. (2017) Nat. Methods, 14, 593-599), is used as described herein. In this way, the cell size becomes ˜40-160 μm which is the same order as the feature size of, for example, Slide-seq arrays. Importantly, diffusion is unaffected by this process and therefore the methods provided herein improve both relative and absolute resolution.

In various embodiments, gel expansion comprises a gel precursor solution, e.g. acrylamide, which is applied to the tissue sample in a high salt buffer and then cross-linked in place. Next, the high-salt buffer is exchanged for pure water which causes the gel to expand. In various embodiments, the gel expands approximately 1, 2, 3, 4, 5, 6, 7, 8, 9 or 10-fold. In one embodiments, the gel expands approximately 4-fold. This process can be repeated to achieve a 16 fold expansion. Instead of letting the RNA diffuse to the spatial barcodes on the slides, the methods provided herein release the barcodes from the slide and let it diffuse to the RNA. This “swap” of the mobile nucleic acid fraction is necessary due to the sample fixation in gel expansion. This swap further provides the added benefit of a more uniform diffusion process because diffusion is dependent on molecule size and RNA transcripts can vary in size between 500-1,0000 bp, while all spatial barcodes are, in some embodiments, small and of identical size. In some embodiments, of the present disclosure, allowing the spatial barcodes to diffuse provides the additional benefit that genomic DNA features such as ATAC-seq and chromosomal polymorphisms can be resolved spatially.

Thus, in one embodiment of the present disclosure, single-cell resolution using, for example, Slide-seq with minimal changes to the hardware and only minimally extending sample handling on the user side is provided. The present methods enable measuring the same genomic modalities that can be profiled with droplet microfluidics at spatially resolved single cell resolution: immunophenotype with DNA-tagged antibodies, ATAC-seq, single-cell amplicon sequencing, single-cell RNA-seq, among others known in the art.

In another embodiment of the present disclosure, spatially-resolved genomics is enabled through a specialized sample preparation which serves as an input for droplet microfluidic workflows. By way of example, in one embodiment a tissue section is placed over a spatial index array and embedded (thickness 10-100 um) in a photopolymerizable gel precursor mix, such as acrylamide/N,N′-Bis(acryloyl) cystamine with a photo-initiator such as riboflavin (vitamin B2). In one embodiment, the gel is dissolvable. The tissue is overlaid with a patterned photomask (e.g., a honeycomb lattice) with feature size in the range 1-100 um. Ultraviolet light (UV) is then used to polymerize the hydrogel precursor. Exposure to UV leads to a tessellation of the tissue into homogeneous hydrogel particles with feature size determined by the thickness and the photomask pattern. In one embodiment, the UV exposure releases the spatial indexes (e.g., a first binding moiety comprising a spatial barcode molecule) simultaneously, which diffuse into the hydrogel where they are immobilized. In another embodiment, the tissue is embedded in a hydrogel prior to the application to the spatial array described above, and UV exposure locally dissolves the hydrogel to form the tessellation.

The hydrogel particles, each containing a fraction of the tissue and spatial barcode sequences indicating the spatial position, can then be collected and loaded into microfluidic droplets together with a second barcoded bead (e.g., a second binding moiety comprising a sequence barcode) according t ovaries embodiments of the present disclosure. This step is analogous to common single-cell workflows where cells are paired with a barcode bead.

According to one embodiment of the present disclosure, the spatially encoded cell containing particles are paired with barcoded beads. In consequence, the downstream process is analogous to established single-cell workflows which yields for each tissue hydrogel a barcoded library of its nucleic acid content (can be RNA, genomics, ATAC-seq, DNA coded antibodies, etc.) together with the barcoded spatial indexes. After sequencing, the data is de-convoluted into individual tissue fragments through the microfluidic barcode (e.g., sequence barcode) and the barcoded spatial indexes (e.g., spatial barcode or “zipcode” as described herein) reveal the position of each fragment within the original tissue section.

Thus in various embodiments, the present disclosure provides a significant improvement to the current Slide-seq methodology in terms of spatial resolution for RNA-seq experiments. The present disclosure for the first time provides a method that enables spatially-resolved genotyping and ATAC-seq at high resolution and combinations of several different readouts (multiomics).

Gel expansion has previously only been used in context of optical microscopy (Zhang, C., et al., (2020) Curr. Protoc. Neurosci., 92). In optical microscopy the smallest feature size that can be distinguished is fundamentally limited by the wavelength of light, which is constant. By expanding the specimen as described herein, the relative resolution can be increased (4× expanded specimen yields 4× higher spatial resolution). In spatial transcriptomics, the resolution is fundamentally limited by macromolecular diffusion, and thus by increasing the specimen the present disclosure, in various embodiments, improves the relative resolution of the measurement.

Hydrogel-embedded cells have been used previously in specialized microfluidic applications, for example, to profile single cell chromosomal DNA (Lan, F., et al., (2017) Nat. Biotechnol., 10.1038/nbt.3880). Hydrogel embedded tissue has also been minced and used as input for low resolution (millimeter resolution) spatial microbiome profiling of a mouse gut (Sheth, R. U., et al., (2019) Nat. Biotechnol., 37, 877-883). To the contrary, the present disclosure provides various methods that use a photo-patterning process and spatial indexes (e.g., spatial barcodes) to provide a homogeneous sample of gel-embedded cells (or other biomolecules described herein) which can be used as input for single-cell microfluidic workflows.

As described herein, in various embodiments the methods provided herein enable spatially-resolved whole transcriptome sequencing of tissue sections with up to single-cell resolution and with minimal lab equipment. Droplet microfluidics does not resolve spatial organization. Optical microscopy with hybridization probes is only available to expert labs, requires expensive equipment and can only probe up to a few hundred transcripts simultaneously. In situ sequencing can probe the whole transcriptome but detection efficiency is low, it is even more challenging than hybridization and relies on at least equally expensive equipment. Slide-seq without this invention provides spatial resolution of order ˜100 μm well short of single cell resolution.

The photo-patterned methods provided herein additionally achieves the goal of single-cell resolved spatial genomics. Spatial transcriptomics at high throughput is currently orders of magnitudes from single cell resolution and other modalities such as ATAC or chromosomal DNA has, until now, not been demonstrated in spatial high throughput workflows.

As described herein, a major advancement provided by the methods of the present disclosure is the ability to transform a 2D sample (e.g., a tissue sample) into a sample that can be (1) in a suspension and (2) sequenced in a sequencing machine.

As used herein, the term “sample” or “biological sample” or “tissue sample” encompasses a variety of sample types obtained from a variety of sources, which sample types contain biological material. For example, the term includes biological samples obtained from a mammalian subject, e.g., a human subject, and biological samples obtained from a food, water, or other environmental source, etc. The definition encompasses blood and other liquid samples of biological origin, as well as solid tissue samples such as a biopsy specimen or tissue cultures or cells derived therefrom and the progeny thereof. The definition also includes samples that have been manipulated in any way after their procurement, such as by treatment with reagents, solubilization, or enrichment for certain components, such as polynucleotides. The term “sample” or “biological sample” encompasses a clinical sample, and also includes cells in culture, cell supernatants, cell lysates, cells, serum, plasma, biological fluid, and tissue samples. “Sample” and “biological sample” includes cells, e.g., bacterial cells or eukaryotic cells; biological fluids such as blood, cerebrospinal fluid, semen, saliva, and the like; bile; bone marrow; skin (e.g., skin biopsy); and viruses or viral particles obtained from an individual.

As described more fully herein, in various aspects the subject methods may be used to detect a variety of components from such biological samples. Components of interest include, but are not necessarily limited to, cells (e.g., circulating cells and/or circulating tumor cells), viruses, polynucleotides (e.g., DNA and/or RNA), polypeptides (e.g., peptides and/or proteins), and many other components that may be present in a biological sample.

The terms “polynucleotide” and “nucleic acid” and “target nucleic acid” refer to a polymer composed of a multiplicity of nucleotide units (ribonucleotide or deoxyribonucleotide or related structural variants) linked via phosphodiester bonds. A polynucleotide or nucleic acid can be of substantially any length, typically from about six (6) nucleotides to about 10⁹ nucleotides or larger. Polynucleotides and nucleic acids include RNA, cDNA, genomic DNA. In particular, the polynucleotides and nucleic acids, is used herein to refer to a binding moiety used in the methods described herein and/or as a target of the methods described herein (e.g., a target whose location and sequence is determined by practicing the methods described herein).

The term “oligonucleotide” refers to a polynucleotide of from about six (6) to about one hundred (100) nucleotides or more in length. Thus, oligonucleotides are a subset of polynucleotides. Oligonucleotides can be synthesized manually, or on an automated oligonucleotide synthesizer (for example, those manufactured by Applied BioSystems (Foster City, CA)) according to specifications provided by the manufacturer or they can be the result of restriction enzyme digestion and fractionation.

The term “primer” as used herein refers to a polynucleotide, typically an oligonucleotide, whether occurring naturally, as in an enzyme digest, or whether produced synthetically, which acts as a point of initiation of polynucleotide synthesis when used under conditions in which a primer extension product is synthesized. A primer can be single-stranded or double-stranded.

The term “nucleic acid array” as used herein refers to a regular organization or grouping of nucleic acids of different sequences immobilized on a solid phase support at known locations. The nucleic acid can be an oligonucleotide, a polynucleotide, DNA, or RNA. The solid phase support can be silica, a polymeric material, glass, beads, chips, slides, or a membrane. The methods of the present invention are useful with both macro- and micro-arrays.

The term “protein” or “protein of interest” (e.g., as it relates to a target biomolecule) refers to a polymer of amino acid residues, wherein a protein may be a single molecule or may be a multi-molecular complex. The term, as used herein, can refer to a subunit in a multi-molecular complex, polypeptides, peptides, oligopeptides, of any size, structure, or function. It is generally understood that a peptide can be 2 to 100 amino acids in length, whereas a polypeptide can be more than 100 amino acids in length. A protein may also be a fragment of a naturally occurring protein or peptide. The term protein may also apply to amino acid polymers in which one or more amino acid residues is an artificial chemical analogue of a corresponding naturally occurring amino acid. A protein can be wild-type, recombinant, naturally occurring, or synthetic and may constitute all or part of a naturally-occurring, or non-naturally occurring polypeptide. The subunits and the protein of the protein complex can be the same or different. A protein can also be functional or non-functional.

Generally, other nomenclature used herein and many of the laboratory procedures in cell culture, molecular genetics and nucleic acid chemistry and hybridization, which are described below, are those well-known and commonly employed in the art. (See generally Ausubel et al. (1996) supra; Sambrook et al, Molecular Cloning: A Laboratory Manual, Second Edition, Cold Spring Harbor Laboratory Press, New York (1989), which are incorporated by reference herein). Standard techniques are used for recombinant nucleic acid methods, polynucleotide synthesis, preparation of biological samples, preparation of cDNA fragments, isolation of mRNA and the like. Generally enzymatic reactions and purification steps are performed according to the manufacturers' specifications.

“Detecting” or “determining” as used herein generally means identifying the presence of a target, such as a target nucleic acid or protein or biomolecule. In various embodiments, detection signals are produced by the methods described herein, and such detection signals may be optical signals which may include but are not limited to, colorimetric changes, fluorescence, turbidity, and luminescence. Detecting, in still other embodiments, also means quantifying a detection signal, and the quantifiable signal may include, but is not limited to, transcript number, amplicon number, protein number, and number of metabolic molecules. In this way, sequencing or bioanalyzers are employed in certain embodiments.

According to some embodiments of the present disclosure, a lysing reagent is used in the detection methods. Lysing agents may include, for example chemical lysis, such as SDS, detergents, alkaline, and acid; biological lysis, such as lysis enzymes, viruses, and phages; and physical lysis such as beads beating, grinding, frozen-thaw, and sonication, heating, cutting, and laser or ion beams.

The present disclosure provides methods of detecting a target in a sample, where the target may be, for example, a nucleic acid (RNA, DNA), biomolecules such nucleic acids, genes, proteins or polypeptides or epitopes, as well as biological particles such as cells (bacterial, human, parasite) and viruses.

As used herein, “biomolecules” can be nucleic acids themselves or can be other biomolecules that are associated with nucleic acids or comprise nucleic acids such as cells, proteins, or nuclei and the like. The term “substrate” or label substrate” or labelling substrate” includes, without limitation, a slide or an array or, in one embodiment can be the tissue sample itself.

As used herein, the term “embedding gel precursor solution” means a solution of monomeric molecules or combinations of reactive monomeric molecules in water or other polar solvents such as DMSO, DMF, EtOH, MeOH, and acetonitrile and mixtures thereof, which can be converted into a hydrogel through polymerization using known techniques. In particular, molecules containing an acryl moiety which can be polymerized through radical polymerization, such as acrylamide, methacrylamide, N-isopropylacrylamide and methacrylic acid, N,N′-Methylenebisacrylamide, N,N′-Bis(acryloyl)cystamine and derivatives are contemplated herein in various embodiments. In some embodiments, embedding also comprises polymerization of molecules through aldehyde moieties such as formaldehyde, glutaraldehyde. In other instances some or all of the building blocks are polymers, for example linear or branched molecules of the polyethylene glycol (PEG) family, sugars, peptides, nucleic acids. Still other polymers are soluble in polar solvents and can be crosslinked through reactive moieties introduced within or at their ends, such as acrylic moieties, amines, azides, carboxylic moieties, aldehydes, thiols, thiolates, peptides, moieties suitable for click chemistry. The methods provided herein also comprise gel formation through physical aggregation of preformed polymers, such as cooling a solution of melted agarose or alginate crosslinking through electrostatic interaction with ions, in particular divalent cations. As used herein embedding also refers to the use of reactive molecules such as aldehydes, maleimides, succinimides that can be used to crosslink biological structures such as proteins, nucleic acids and lipids in tissues to form a hydrogel.

As used herein, the phrase “dissolvable hydrogel” means a hydrogel formed through any of the above mentioned mechanisms that can be back converted into a non-crosslinked state through application of a suitable stimulus. In various embodiments the stimulus may be, for example, a light pulse, metal chelating molecules like EDTA, EGTA to dissolve alginate gels, a reducing agent or acids and bases capable of hydrolizing disulfide bonds (e.g. N,N′-Bis(acryloyl)cystamine), or heat. The phrase may also refer to hydrogels containing chemical bonds that can be broken through irradiation with light of the appropriate wavelength. For example, hydrogels formed with 5-(meth-acryloyloxy)-2-nitrobenzyl methacrylat or 7-(hydroxyethoxy)-4-methyl-coumarin as crosslinker are contemplated. Local melting of a thermally responsive hydrogel such as agarose with infrared light is also contemplated herein as another example of dissolving.

As used herein, the phrase “photopolymerizable hydrogel precursor” refers to a hydrogel precursor as defined above, including dissolvable hydrogels, for which hydrogel formation through polymerization or through crosslinking of preformed polymers can be controlled through, for example, exposure to light of the appropriate wavelength. By way of example, a solution comprising acrylamide and or methacrylamide, a crosslinker such as N,N′-Methylenebisacrylamide, N,N′-Bis(acryloyl)cystamine and a photoactive radical donor such as riboflavin is provided herein.

As used herein, the phrase “photopolymerizable, dissolvable hydrogel precursor” refers to a hydrogel precursor as defined above which forms a hydrogel that has the properties of a hydrogel defined as dissolvable hydrogel.

The terms “lattice” and patterned photomask” as used herein refer to a device that enables modulating the exposure of a substrate to electromagnetic waves, electron or ion beams in a spatially controlled manner. In one embodiment it refers to a device that can modulate the exposure of infrared, visible and UV light. In one embodiment, a method disclosed herein uses a photomask as a device that shades certain parts of the substrate thereby locally preventing UV exposure. In another embodiment the photomask refers to a device which modulates light exposure through interference, such as an optical grid . . . .

As used herein the term “expansion gel precursor solution” means in some embodiments a gel precursor solution that can be converted into a hydrogel as described herein and wherein the hydrogel shows swelling upon solvent exchange. In one embodiment, hydrogels that expand isotropically and exhibit a volumetric change of at least 1.5 fold are provided herein. In some embodiments, PEG and acrylamide based hydrogels are provided herein.

In various embodiments of the present disclosure, a tissue sample is processed and prepared under conditions that allow gelation of the tissue sample, expansion of the tissue sample, or both gelation and expansion of the tissue sample. “Gelation of the tissue sample,” as sued herein, refers to any suitable treatment of the tissue sample known in the art that causes the tissue to be embedded in a hydrogel. In one embodiment it refers to exposing the tissue to molecules capable of permeating the tissue and subsequently polymerizing into a hydrogel. Said molecules are preferably acrylic molecules and more preferably acrylamide. The term “a gel scaffold of the (embedded) . . . tissue sample” refers to a polymer mesh that surrounds and penetrates the original tissue structure after gel embedding.

The present disclosure provides numerous methods for acquiring spatial genomic data at a single cell level.

In one embodiment, a method for obtaining both spatial information and sequencing information from a tissue sample is provided comprising (a) preparing a tissue section or sample of 0.1 to 1000 micron thickness, (b) preparing a label substrate with spatial barcodes, (c) applying the tissue section to the label substrate, (d) disaggregating the tissue section to create a suspension, (e) barcoding the suspension with sequence barcodes, (f) sequencing the barcoded suspensions, and (g) bioinformatically inferring/determining spatial position from the barcode combinations.

With respect to steps (a)-(g), the steps are not necessarily in order of execution (e.g., step (a) can occur after step (b), step (f) and step (g) can occur concurrently or sequentially, and so on. Various embodiments of each of steps (a)-(f) are provided herein.

By way of example, in some embodiments in step (a), the tissue sample is frozen and/or sliced, optionally gel-embedded and/or expanded by gel expansion approaches to increase resolution of imaging and labeling with label substrate; the tissue is fixed and sliced or sliced fresh. In some embodiments, the tissue section is: pre-processed and analyzed using antibodies, oligos, methylation, tagmentation; printed zipcodes (i.e., spatial barcodes), dyes, etc.; printed with barcode beads (e.g., as additional zipcodes (i.e., spatial barcodes); pre-digested (in fixed, expanded or gelled); pre-analyzing using imaging, spectroscopy, MS, enzyme assays or FISH analyses.

By way of example, in some embodiments in step (b), the present methods contemplate: creating an array with distinct spatial barcodes or spatial barcode combinations at different positions, where the spatial barcodes are nucleic acids, proteins, peptides, antibody-DNA conjugates, isotopes, MS detectable dyes, DNA-conjugates, etc.; spatial barcodes with UMIs to enhance quantitativeness for accurate concentration estimation; creating an array through printing (droplet, photo initiated synthesis); creating an array from pre-made barcode particles; and creating an array by bridge PCR; creating an array with a recognizable gradient. The present methods optionally also include: creating an array with multiple functional groups per spatial barcode, for example A-type and B-type that form duplexes that sequence and allow determination of neighboring pairs, or fixed+mobile oligos such that mobile bind to tissue and fixed capture tissue-released material, to enable parallel analysis.

By way of example, in some embodiments in step (c), the methods provided herein include: bringing into close contact to label substrate, contacting the tissue section to one or multiple label substrates (sandwich), and where label substrates may be differently configured for different tasks, such as mobile versus fixed target capture; gelling including polymerization, crosslinking, and fixation, high viscosity (glass), patterning to enabling facile gel disaggregation and/or to enable digesting; optionally releasing biomolecules by digesting cell walls, proteins, etc. from tissue, substrate, cell nuclei, bodies, or nucleic acids and binding to substrate spatial barcodes; generating gradient patterns of labels in the tissue section such that neighboring labels commingle to generate specific patterns which can be interpreted bioinformatically to triangulate cell position and to identify spatial barcode locations; adding additional labels to the substrate; and optionally analyzing the labeled tissue and/or substrate for imaging, MS, and/or spectroscopy.

By way of example, in some embodiments in step (d), the methods provided herein include: extracting nuclei or cells (e.g., digestion); grinding/slicing into a particulate suspension; melting or hydrolysis or disaggregation of gels or tissues (e.g., photo cleavage, chemical, spatial, global, and targeting specific gel types in multi-gelled tissue section).

By way of example, in some embodiments in step (e), the methods provided herein include: labeling the suspension, e.g., using probes, antibodies, nucleic acids, etc; analyzing the suspension using, for example, FACS, imaging, spectroscopy, MS; sorting, for example by selecting specific tissue sections or tissue section samples or cells or nuclei; and/or barcoding the suspension.

By way of example, in some embodiments in step (f) and/or in step (g), the methods provided herein include: sequencing, MS, droplet assays, etc. of suspension phase particles; linking or inferring spatial position from the set of particle associated spatial barcodes, e.g., with a priori knowledge of barcode spatial coordinates or without a priori knowledge (and wherein location is inferred from the sequenced indexes (e.g., sequence barcodes); where spatial barcodes may exist within a single read (A-B type) or different reads of the same barcode cluster or where diffusion kinetic modeling used to enhance positional information or where triangulation achieved through co-occurrence of multiple zipcodes within one barcode cluster; gel expansion gel expansion is performed to enhance spatial resolution of the entire process, and computational analysis used to infer resolution and chuck locations based on expansion modeling; and/or multimodal analysis including, for example, linking ABseq, DNAseq, RNAseq, imaging, FACS.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “and,” and “the” include plural referents unless the context clearly dictates otherwise. Thus, for example, reference to “a conformation switching probe” includes a plurality of such conformation switching probes and reference to “the microfluidic device” includes reference to one or more microfluidic devices and equivalents thereof known to those skilled in the art, and so forth. It is further noted that the claims may be drafted to exclude any element, e.g., any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible. This is intended to provide support for all such combinations.

### Example 1

Determining Spatial and Genomic Information from a Tissue Sample

This Example provides a method for sequencing a tissue section that allows spatial information about the tissue to be recovered by sequencing approaches, such as mass spectrometry and nucleic acid sequencing.

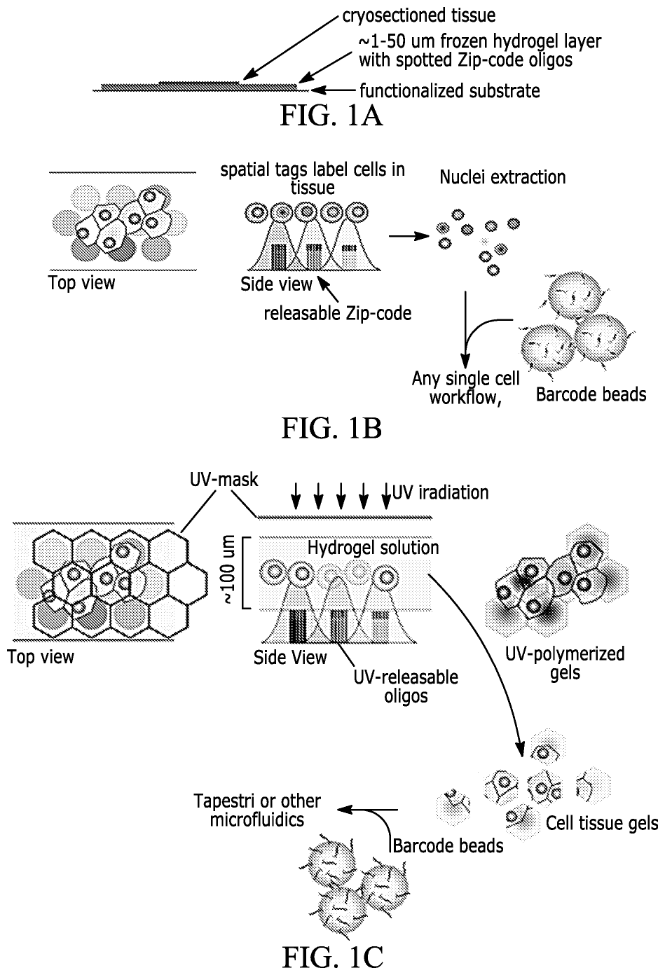

FIG. 1 shows one embodiment for an experimental setup of a label substrate with a cryosection of a tissue. A dry hydrogel layer is spotted with “zipcode” oligos in known spatial positions. The hydrogel substrate is wetted with a mild cell lysis buffer and frozen. A frozen cryosection is placed over the Zipcode array and thawed to transfer the Zipcodes into the cells. Once thawed, the Zipcode DNA oligos diffuse into the tissue and label cells and cell nuclei in close proximity. These cells or nuclei can then be extracted and evaluated with any single-cell method (FIG. 1B). FIG. 1C provides an exemplary workflow with gellation. The tissue section is embedded in a hydrogel matrix and applied to the label substrate. A photomask is used to polymerize the gel embedded tissue into micron sized gel chunks and simultaneously release the zipcode oligos to diffuse into the gel chunks in close proximity. The gel chunks can then be processed like cells with any single cell.

Thus, an exemplary method comprises preparing a tissue section of micron scale and applying it to a label substrate. The label substrate comprises oligos or affinity moieties that can attach to the tissue section or accept molecules released from the tissue section, thereby labeling the tissue section with mobile “zipcode” moieties (e.g., spatial barcodes) or attaching to the substrate molecules from the tissue section to fixed zipcode moieties. The tissue section can be gelled, before, during, or after attachment to the label substrate, using chemical or photo methods. The gelation approach is selected to facilitate disaggregation of the tissue section, for example, by allowing boundaries between spatial sections of the gelled tissue section to be dissolved. The label substrate and labelled tissue section can be analyzed and further processed, if desired, and subjected to disaggregation to prepare a suspension. Once in suspension, the tissue section chunks can be further processed to label with affinity molecules or analyzed, for example by imaging, and subjected to barcoding (e.g., with sequence barcodes) using a variety of methods. After barcoding, the materials from the tissue chunks, such as nucleic acids, can be analyzed by barcode sequencing, thereby providing sequence information that can be used to infer the original locations of the chunks in the tissue section based on combinations of barcode and zipcode sequences.

In various embodiments, methods for sequencing a tissue section that allows spatial information about the tissue to be recovered by sequencing follow the following workflow, noting the described flexibilities.

Preparing a Tissue Section of 0.1 to 1000 Micron Thickness

A tissue section of 0.1 to 1000 micron thickness is prepared for example by freezing and/or slicing the tissue into layers. The tissue can be processed as is, or subjected to additional processing to facilitate analysis. For example, the tissue can be perfused with matrix molecules that facilitate gel expansion, to enable high resolution microscopy or barcoding, for highly spatially resolved single cell sequencing. Tissues may also be fixed or sliced fresh. Components from the tissues may be captured onto the perfused matrix, such as nucleic acids, lipids, small molecules, sugars, proteins, etc.

They may also be subjected to further processing, for example, to label with probes, such as antibodies or fluorescent oligonucleotides. Tissue sections may be printed with components, such as oligonucleotides, dyes, and beads, to facilitate downstream analysis. For example, in one embodiment, printing of randomly labeled beads can be used to spatially label tissue sections based on a broadcasting triangulation concept described later. Tissue components can be subjected to chemical or enzymatic processing to facilitate additional analysis, such as to enable methylation or other epigenetic sequencing. For example, tagmentation can facilitate amplification of sequences for library preparation, or to perform ATAC seq analysis. Enzymes and other components can facilitate lysis, such as cell wall digesting agents when applied to certain cell types, such as fungi or microbes, or proteases to facilitate digestion of cell components, such as chromatin. Tissues may be processed by printing materials on them using available modalities, such as zipcodes, dyes, chemicals, etc. using inkjet, laserjet, and dispensing technologies.

These steps can be done on fixed, fresh, frozen, gelled, or expanded tissue sections. For example, in one embodiment, to facilitate highly resolved single cell methylation DNA sequencing, tissues can be perfused with gel matrix, expanded, and processed with bisulfate. The prepared tissues can be analyzed, before, during, or after processing. For example, imaging spectroscopy and mass spectrometry can be used to obtain information about the sample in its spatial context. The sample may be subjected to fluorescence in situ hybridization analysis (FISH) to generate FISH based images that can facilitate spatial sequencing of the tissues by combining FISH with other forms of sequence analysis, such as barcode based sequencing. This can be accomplished, for example, by matching FISH patterns with patterns identified in the barcode sequencing data.

Preparing a Label Substrate

To enable spatial sequencing, a label substrate (e.g., labelling substrate) is required that is patterned with zipcode molecules at different locations. The zipcodes can comprise nucleic acids, proteins, peptides, antibody-DNA conjugates, mass spec detectable dyes, etc. These components can be attached to the substrate through various methods, such as inkjet printing or array based synthesis by photo-initiated chemistries, as are used to make microarrays. Arrays may be generated by constructing barcode particles applied as a single or multilayer on the substrate. Bridge PCR can generate zipcode colonies of nucleic acids that share the same sequence, in close spatial proximity. Arrays may be generated with recognizable gradients in the applied zipcodes.

For example in one embodiment, droplet printing can deposit specific zipcodes that will dry into a recognizable pattern, such as a “coffee ring” effect, in which the concentrations of oligos at different locations in the dried drops follows a recognizable pattern. This pattern can be used in the sequence data to enhance inferred positional accuracy of the zipcode location on the substrate, and tissue chunks expressing that zipcode. The zipcodes may be attached to the substrate such that they are releasable or fixed, at different steps in the workflow. For example this can facilitate binding of zipcodes to tissue sections, or binding of tissue section components to fixed barcodes on the substrate. Stimuli may be applied to facilitate release of zipcodes, such as chemical agents that break a linkage bond to the substrate.

Photo methods can be used to facilitate release, and photo-patterning to target release at specific locations. Substrates can be prepared with overlapping zipcodes that can be of similar or distinct type. For example, binding moieties can overlap with mobile zipcodes, such as antibodies to capture epitopes released from tissue sections, and zipcode oligonucleotides to bind to tissue. Zipcodes can be designed to facilitate spatial determination. For example, zipcodes can comprise “A”, “B”, “C”, etc. types that are partially complementary such that, upon release neighboring sequences can anneal and integrate into a single read. Based on conjugation of types into chimeric reads, neighbor locations of zipcodes can be inferred. Zipcodes can be partly mobile or fixed, with similar or different sequences used for each, allowing binding to or from tissue sections to perform parallel analysis of different molecule types either on the substrate or in the later suspension phase.

Applying and Labeling the Tissue Section

Tissue sections are labeled by placing them in proximity to the label substrate, such that elements from the tissue and/or substrate commingle. Multiple label substrates may be placed into contact with a tissue section, or multiple tissue sections onto multiple label substrates. For example a label substrate can be placed “above” and “below” a tissue section to generate a “sandwich” structure to facilitate analysis, such as to enable greater resolution or to specifically target different components by the two label substrates. Tissues may also be placed so as to span multiple label substrates either above, below, or above and below.

In some embodiments, it is desirable to gel the tissue section either before, during, or after contact with the label substrates. Gelation can be accomplished via a number of techniques, such as polymerization, crosslinking, or fixation. Alternatively, the tissue section can be “solidified” by increasing its viscosity such that it is not technically a gelled material, but has a solid like character, such as viscous liquid or glass. Phase change of the tissue section can be accomplished using a variety of initiation techniques, such as photo or chemical initiation. Phase change can be initiated by transmitting light through the tissue section, or by release of molecules from a contacting substrate. Phase change can be performed globally on a large portion of the tissue section, over local regions, or with micro-patterning.

Multiple matrices can be introduced to facilitate controlled patterning to facilitate further analysis. For example in one embodiment, a mixture of gel matrices can be used, one photo polymerizable and the other not, the photopolymerizable one being elastic, and the other being meltable, such as polyacrylamide and agarose, respectively. Upon contact with the substrate and processing, a light pattern can be applied to photopolymerize a close packed grid of solid regions interspaced by liquid regions. The entire substrate can be cooled, gelling the agarose and thus the tissue section. The now gelled tissue section can be recovered from the substrate and heated to melt the agarose components, with the polyacrylamide regions remaining solid since they do not melt with application of moderate heat. Reagents can be applied to lyse cells or digest interstitial tissue material, to release gels. These gel “chunks” can be processed as a suspension phase using for example single cell sequencing methods to attach barcodes to the tissue sections they encase. A reversible cross linker can be included in the polyacrylamide to facilitate liquefaction of the gel in post processing steps, if desired.

Tissue sections can be subjected to a variety of other processing steps to facilitate spatial analysis. For example, enzymes and other compounds can be added to digest cell walls, proteins, etc, to facilitate access to important molecules such as genomic DNA and RNA. Material can be released from the tissue sections and captured into the surrounding gel or label substrates, or materials can be released from the gel or label substrate to bind to the tissue sections. For example in one embodiment, moieties may be released from the label substrate to bind to the nuclei, cell bodies, or nucleic acids of the tissue section, for example to attach zipcode sequences for downstream detection. Various stimuli can be used, such as rehydration, catalytic cleavage, photo cleavage, thermal, redox, pH cleavage, and so on. Upon release of molecules to or from a tissue section or label substrate, gradient patterns can be generated that can be modeled to enhance spatial determination. For example, in one embodiment, zipcode sequences from neighboring spots may commingle and form hybrids with which to infer neighbor graphs. These graphs can be used to determine the locations of zipcodes retrospectively, without having to pre-identify them. In addition, this information can be combined with pre-identified locations to ensure quality control and accuracy. Modeling of gradient patterns detected by imaging and/or sequencing analysis can be used to interpolate higher resolution locations of specific target molecules within the sample. For example in one embodiment, an RNA molecule residing at a location in a cell may be bound by multiple zipcode sequences, allowing estimation of zipcode concentrations at that position. When combined with a model of zipcode mobility, this can be used to estimate a location of the RNA.

A similar analysis can be applied to a cell body, nuclei, or solidified chunk of tissue section, enabling triangulation similar to radio triangulation of a signal broadcast. This can be applied in reverse as well, to provide accurate locations of zipcodes “broadcasting” from specific spots on the label substrate or gel. In addition, if the tissue section is printed with random beads, these beads can release an oligo to the surrounding tissue matrix that can label the tissue in a way similar to the label substrate. Due to chemical mobility of released zipcodes from the beads, a “halo” of oligo will result around each bead, allowing bead position to be inferred by reverse triangulation in the sequencing data, in a similar process applied to the label substrate. These methods can be combined, applying substrate and bead labels, to multiply-barcode a sample to allow targeting of distinct information or enhancement of spatial and sequencing accuracy. Tissue sections that have been subjected to labeling can be further processed with additional labeling from bulk solutions, to label important components. For example, tissue sections can be labeled with additional antibodies, oligos, etc, to enable detection by imaging or barcode based sequencing. This can be applied to tissues either while in contact with the label substrate, or after release. For example in one embodiment, a tissue section, either before, during, or after substrate labeling, can be further labeled with fluorescent oligos to perform FISH analysis. The resultant fish data can be used to infer locations of specific nucleic acids and/or cells in the tissue section that can be additionally sequenced. Probes targeting zipcode sequences can also be applied. Patterns determined by FISH can be matched with ones obtained by sequencing to provide additional spatial resolution to the sequence data.

Disaggregating the Tissue Section to Create a Suspension

Processed tissue sections can be disaggregated to facilitate analysis. For example nuclei or cells can be recovered from processed tissue sections and subjected to “single cell genomics” processing, such as “barcoding” with droplets, wells, etc. Processed tissue sections can be physically disaggregated for example by grinding into microscale chunks appropriate for single cell genomics workflows. Processed tissue sections infused with gel may be disaggregated by exploiting gel reversibility, such as by melting gels with thermal control, or reversing crosslinks by application of light or chemicals. This can be done to fully remove gel matrix solidification, or boundaries between adjacent gels. The tissue material between the solid gels can then be subjected to digests to release gel chunks that can then be subjected to single cell genomic workflows. The culmination of these steps is to transform the once intact tissue section into a suspension appropriate for analysis by single cell genomic workflows, which typically require microscale suspensions with entities between 0.1 and 1000 microns in diameter.

Processing and Analyzing the Suspension

Tissue section suspensions must be further processed to allow spatial sequencing analysis. For example, in one embodiment, nuclei, cell bodies, or gelled tissue section chunks can be labeled with antibodies, oligonucleotides, etc. Once labeled, they can be subjected to various analyses, such as flow cytometry, imaging, spectroscopy measurements, and mass spectrometry or cytometry. These analyses may be partly or wholly destructive, if necessary, in some embodiments, reserving a plurality of entities for sequencing. Suspended entities may also be subjected to sorting to target specific entities for analysis. For example, markers that label entities applied either prior to, during, or after disaggregation may be used to detect specific suspension entities, which can then be used to trigger isolation by sorting. Isolation can dispense entities into wells for single analysis, or combine them into a new subset suspension that is compatible with barcode based single cell genomics analysis. Once a suitable suspension is obtained, it can be subjected to single cell genomic analysis. For example, nuclei, cell bodies, or gel chunks can be introduced into barcode approaches, such as microwells or droplets, to label their components with barcode sequences. Close packed encapsulation can facilitate loading of droplets, while size exclusion in defined microwells matched with nuclei, cell body, or gelled chunks can efficiently load wells. Combinatorial tagmentation can apply barcodes in a bulk suspension process. The resulting barcoded chunks can be processed using usual single cell genomic methods to for example sequence DNA, RNA, and oligonucleotides associated with affinity reagents, such as antibodies or lipids.

Analyzing Barcoded Suspensions

The result of the previously described processing steps is to generate a multi-parametric dataset comprising sequencing, imaging, spectrographic, mass spec etc., data types, of the tissue section intact and/or as a labeled suspension phase. This incorporates spatial information based on pixel coordinates in the various imaging modalities (e.g. fluorescence, mass spec) or zipcode based sequencing. These modalities are linked in the datasets by the barcodes and zipcodes, and can be thusly used to inform correlations between these distinct modalities applied to the same local regions of tissue sections. For example, in one embodiment, the datasets can first be grouped based on barcode sequence, aggregating together all sequence information associated with a given chunk of tissue section.

All zipcode sequences within that barcode cluster can then be identified, to locate that chunk in the original tissue section. The inferred location can be compared with data from the same pixel location in the various captured images, thereby linking the imaging and sequence data. This can follow a recursive process where first zipcodes are identified, and a graph of all zipcodes generated to identify zipcode positional coordinates on the substrate, and those coordinates annotated to a given barcode cluster. If gel expansion is not used, this will relate to the native location of the tissue section at its original size. If expansion is used, the data will correspond to features that were originally closer together in the native tissue, the result being an enhancement in spatial resolution. As mentioned previously, zipcode locations may be known a priori based on the label substrate manufacture and/or quality control process, or inferred a postieri based on the sequence data and how different zipcodes and barcodes commingle when clustered. Methods of zipcode and/or tissue section components can enhance triangulation of the target location based on a “broadcast” concept and co-occurrence of zipcodes within distinct barcode clusters, which can be used to infer neighbors.

Concentration measurements can be inferred from read counts of a zipcode, which may be labeled with molecular indices to enhance quantitation. Additionally, zipcodes may be designed to facilitate neighbor detection. For example, in one embodiment, complimentary “A” and “B” zipcodes can be used that anneal to one another such that they yield a chimeric read in the data. Thus, identification of chimeric zipcode sequences can in some cases be used to infer that the given zipcodes hybridized at a certain frequency and thus may correspond to neighboring spots. By aggregating together multiple such zipcode chimeras correspond to different neighboring spots, one may triangulate a given zipcodes position with high accuracy. Gel expansion can be implemented to enhance resolution further, if desired. Multimodal datasets comprising DNA, RNA, Antibody sequencing, and imaging, FACS, MS, etc, are generated, where all this information is known for each tissue section entity and, potentially, by implementing FACS and gel expansion, spatial resolutions smaller even than the tissue entity. For example in some embodiments, such an approach would allow location of specific RNAs within a single cell.

### Example 2

Determining Single-Cell RNA Profiles at 200 Um from Mouse Brain Xenograft

This Example provides one application of the described method for sequencing a tissue section at single cell resolution and recovering the spatial origin of each cell.

Label Substrate Generation

The label substrate is constructed by first functionalizing a glass microscopy slide with 3-(trimethoxysilyl)propyl methacrylate (Kang, C.-C., et al., Nature Protocols 11:1508-1530 (2016)). Second, a thin polyacrylamide hydrogel (6% w/v, 19:1 acrylamide:bis-acrylamide) is cast on the functionalized glass slide by mixing acrylamide, bisacrylamide and buffering components in appropriate amounts and initiating polymerization with ammonium persulphate and tetramethylethylenediamine. About 150 μl of the polymerizing pre-gel mix is pipetted on top of the functionalized glass slide and a second non-functionalized glass slide is immediately pressed on top of the solution thus creating a thin liquid film which polymerizes into a hydrogel. After the polymerization is complete, the non-functionalized glass slide is lifted off, leaving the hydrogel chemically bonded to the functionalized glass slide. The gel is rinsed with water and dried.

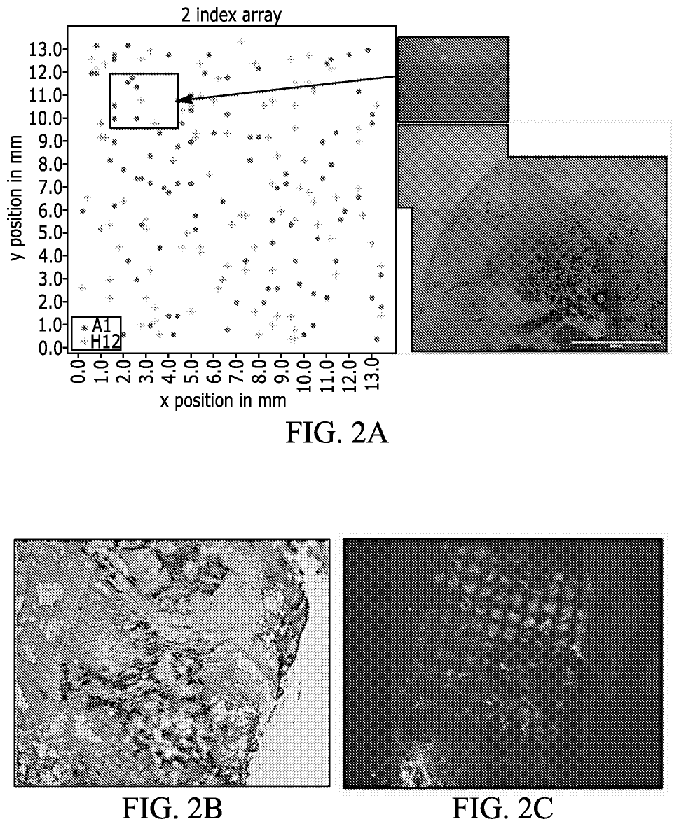

To encode spatial position, a grid structure of unique zipcode DNA oligos is prepared on the dried hydrogel. Each grid position is thereby encoded by a unique pairing of two DNA-barcodes. For instance, with 96 distinct DNA barcodes, 4,560 unique pairs can be formed, enabling the unique encoding of a 67 by 67 grid location. Additional barcode-oligos or a specific spatial organization of the DNA barcode pairs can be used as parity bits. This redundant information can be used to endow the label substrate with error-correcting properties that can help in spatial signal recovery. To produce such a spatial grid structure on the hydrogel, 96 unique zipcode DNA barcodes are diluted to 111M and pipetted into individual wells of a 96-well plate. Additionally, 111M fluorescein is added to two wells to provide a fiducial marker to orient tissue sections on the label substrate (FIG. 2A). A micro array spotting robot (e.g. Scienion sciFLEXARRAYER S3) is used to spot the unique pairs of these 96 barcodes as 80 μm droplets spaced 200 μm apart. FIG. 2A displays a possible encoding, spotting positions of the barcode oligo from well A1 and H12 are given as examples. Next the spotted droplets are dried, the dry hydrogel quickly dipped into a mild cell lysis buffer (10 mM Tris-HCl pH 7.5, 146 mM NaCl, 21 mM MgCl₂, 1 mM CaCl₂), 1% w/v 3-[(3-cholamidopropyl)dimethylammonio]-1-propanesulfonate (CHAPS), 1 mM DTT) and is immediately frozen on dry ice.

Cryo Sectioning of a Mouse Xenograft

A mouse brain with a human cancer xenograft is embedded in optimal cutting density medium (OCT), frozen and mounted on a cryostat. Sections of 12 μm thickness are cut and moved over the frozen label substrate. Next, the label substrate and the superimposed mouse brain section are quickly thawed by putting the finger to the back of the slide and then refrozen and used immediately or stored at −80° C. for later processing.

Zipcode Transfer and Single-Cell Extraction

Thawing of the label substrate with the superimposed tissue sections allows the mild lysis buffer to permeabilize the cell membranes and the zipcode oligos to permeate the tissue by diffusion. FIGS. 2B and 2C shows a tissue which has been removed from a label substrate prepared with fluorescein labeled barcode oligos, demonstrating the barcode transfer into the tissue. This transferred concentration of each barcode oligo into different cells is thus proportional to the distance between the cell location and the spotting locations of each barcode. To ensure an appropriate amount of barcode transfer, the label substrate with tissue was incubated for 5 min on ice before applying a blocking buffer consisting of cell lysis buffer supplemented with 0.1% w/v bovine serum albumin (BSA) and 0.25 mg/ml salmon sperm DNA. The tissue is dissociated and single nuclei are extracted by pipetting up and down in blocking buffer for 2 min, larger chunks are removed by straining through a 40 μm strainer and the nuclei suspension pelleted by centrifugation 500 rcf for 5 min at 4° C. The nuclei are washed in 5 ml phosphate buffered saline supplemented with 0.1% BSA two times, stained with DAPI for counting and re-suspended at a concentration of 1 million nuclei per ml.

Single-Cell RNA Experiment

The nuclei suspension was processed with a 10× Genomics Chromium machine according to the manufacturer's instructions. This process labels each transcript from individual nuclei with an unique single-cell barcode. Since the employed DNA zipcodes carry a hybridization sequence complementary to the secondary primer of the gel beads (GEMs) from the Chromium machine, the zipcode oligos which were successfully attached to individual nuclei receive the same barcode as the corresponding single-cell transcriptome, thus matching the zipcode encoding to the transcriptional state of individual nuclei. After sequencing the single-cell transcriptome library and the zipcode oligo library on a Illumina NextSeq 2000 the single-cell barcodes can be used to link the transcriptional state of individual cell nuclei to their origin within the tissue.

Bioinformatic Analysis

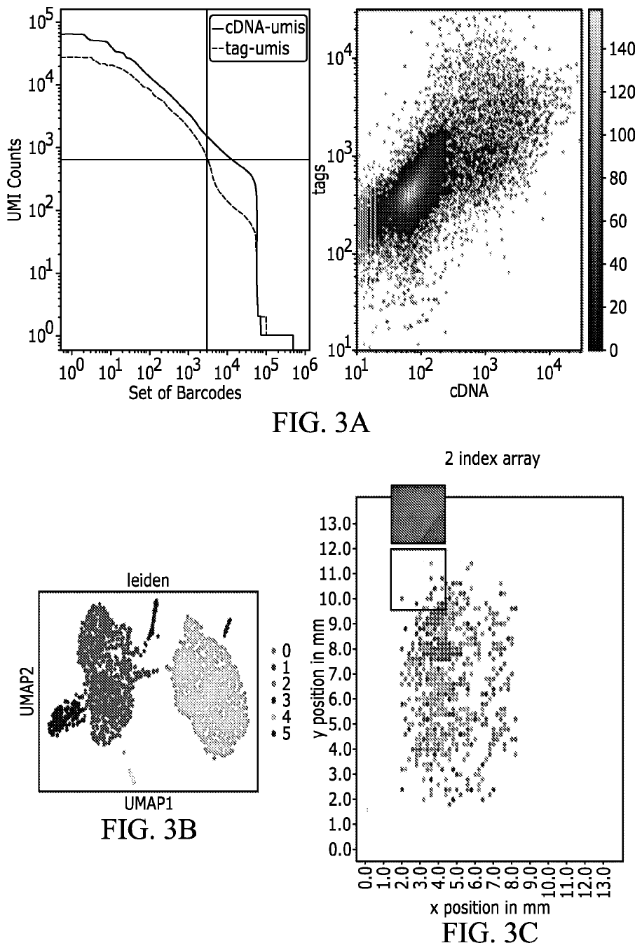

The single cell RNA profiles were analyzed with SCANPY (Wolf, F. A., et al., Genome Biology, 19:15 (2018)) and reveal the expected mouse brain cells (FIG. 3B, clusters 1-4), and the human cancer xenograft (FIG. 3B, cluster 0 and 5)

The spatial origin of each cell can be reconstructed from the zipcode DNA tags in multiple ways. The most simple algorithm counts for each cell barcode the number of occurrence of each spatial barcode, identifies the two most abundant zipcode DNA tags and assigns the cell to the grid spot where this barcode pair has been printed (FIG. 3C, demonstrating spatial organization of the different cell types identified in FIG. 3B. Reconstruction is also in agreement with the brain placement according to the transmission and fluorescence microscopy image—FIG. 3C insert). Alternatively, the zipcode DNA transfer can be modeled by a molecular diffusion in two or three dimensions according to Fick's second law (the heat kernel) or its time integral. Such a model thus provides an estimate for the each zipcode DNA tag concentration at every position of the label substrate. By considering all the measured Zipcode DNA tags of a particular cell barcode as experimental support for any given placement of the cell on the label substrate the most likely origin of each cell can be expressed as maximum a posteriori estimate using Bayes theorem (alternatively the full probability distribution can be calculated). This approach provides the advantage that the tissue structures below the printing resolution can be recovered.

### Example 3

Determining Spatial and Genomic Information from a Brain Tissue Sample

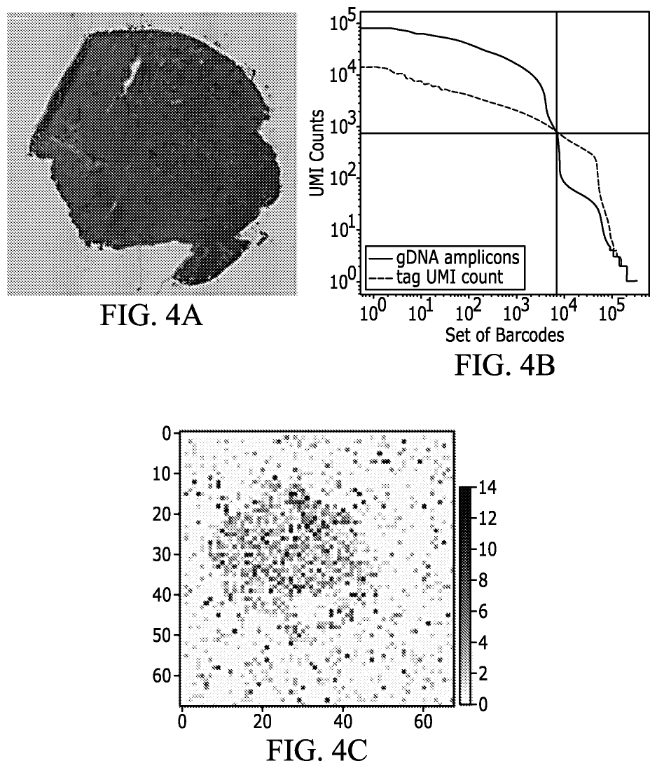

This Example demonstrates spatial analysis of a human melanoma metastasis in combination with single-cell genome mutation detection. The experimental steps for label substrate creation and tissue sectioning, zipcode transfer and nuclei extraction are identical as before, but were performed on a biopsy of a brain metastasis from a melanoma. The nuclei suspension was processed on a MissionBio Tapestri machine (FIG. 4).

The various embodiments described above can be combined to provide further embodiments. All U.S. patents, U.S. patent application publications, U.S. patent application, foreign patents, foreign patent application and non-patent publications referred to in this specification and/or listed in the Application Data Sheet are incorporated herein by reference, in their entirety. Aspects of the embodiments can be modified if necessary to employ concepts of the various patents, applications, and publications to provide yet further embodiments.

These and other changes can be made to the embodiments in light of the above-detailed description. In general, in the following claims, the terms used should not be construed to limit the claims to the specific embodiments disclosed in the specification and the claims but should be construed to include all possible embodiments along with the full scope of equivalents to which such claims are entitled. Accordingly, the claims are not limited by the disclosure.

1. A method of determining the sequence and location of a nucleic acid in a tissue sample, said method comprising the steps of:
(a) preparing a tissue sample, wherein said preparing comprises embedding the tissue sample in an embedding gel precursor solution and contacting the tissue sample with a lattice under conditions that allow gelation of the tissue sample and thereby forming a gelated tissue sample;
(b) preparing a labeling substrate, wherein said labeling substrate comprises a first binding moiety comprising a spatial barcode molecule, wherein said first binding moiety is capable of binding to (i) a biomolecule in the gelated tissue sample; (ii) a biomolecule released from the gelated tissue sample; and/or (iii) a gel scaffold of the gelated tissue sample, and wherein said biomolecule or gel scaffold comprises a plurality of nucleic acid molecules;
(c) incubating the labeling substrate of (b) with the gelated tissue sample of (a) under conditions that allow the first binding moiety to bind to the biomolecule or gel scaffold, thereby labeling the biomolecule or gel scaffold with a spatial barcode;
(d) preparing a suspension solution comprising a labeled biomolecule or labeled gel scaffold from (c) and incubating said solution with a solution comprising a second binding moiety comprising a sequence barcode, under conditions that allow the second binding moiety to bind to a nucleic acid in the labeled biomolecule or labeled gel scaffold of (c), thereby further labeling the biomolecule or gel scaffold with the sequence barcode; and
(e) determining the sequence and location of the nucleic acid.

2. A method of determining the sequence and location of a nucleic acid in a tissue sample, said method comprising the steps of:
(a) preparing a tissue sample, wherein said preparing comprises expanding the tissue sample with an expansion gel precursor solution and thereby forming an expanded tissue sample;
(b) preparing a labeling substrate, wherein said labeling substrate comprises a first binding moiety comprising a spatial barcode molecule, wherein said first binding moiety is capable of binding to (i) a biomolecule in the expanded tissue sample; (ii) a biomolecule released from the expanded tissue sample; and/or (iii) a gel scaffold of the expanded tissue sample, and wherein said biomolecule or gel scaffold comprises a plurality of nucleic acid molecules;
(c) incubating the labeling substrate of (b) with the expanded tissue sample of (a) under conditions that allow the first binding moiety to bind to the biomolecule or gel scaffold, thereby labeling the biomolecule or gel scaffold with a spatial barcode;
(d) preparing a suspension solution comprising a labeled biomolecule or labeled gel scaffold from (c) and incubating said solution with a solution comprising a second binding moiety comprising a sequence barcode, under conditions that allow the second binding moiety to bind to a nucleic acid in the labeled biomolecule or labeled gel scaffold of (c), thereby further labeling the biomolecule or gel scaffold with the sequence barcode; and
(e) determining the sequence and location of the nucleic acid.

3. A method of determining the sequence and location of a nucleic acid in a tissue sample, said method comprising the steps of:
(a) preparing a tissue sample, wherein said preparing comprises (i) embedding the tissue sample in an embedding gel precursor solution and contacting the tissue sample with a lattice under conditions that allow gelation of the tissue sample and thereby forming a gelated tissue sample, and (ii) expanding the tissue sample to form a gelated, expanded tissue sample;
(b) preparing a labeling substrate, wherein said labeling substrate comprises a first binding moiety comprising a spatial barcode molecule, wherein said first binding moiety is capable of binding to (i) a biomolecule in the gelated, expanded tissue sample; (ii) a biomolecule released from the gelated, expanded tissue sample; and/or (iii) a gel scaffold of the gelated, expanded tissue sample, and wherein said biomolecule or gel scaffold comprises a plurality of nucleic acid molecules;
(c) incubating the labeling substrate of (b) with the gelated, expanded tissue sample of (a) under conditions that allow the first binding moiety to bind to the biomolecule or gel scaffold, thereby labeling the biomolecule or gel scaffold with a spatial barcode;
(d) preparing a suspension solution comprising a labeled biomolecule or labeled gel scaffold from (c) and incubating said solution with a solution comprising a second binding moiety comprising a sequence barcode, under conditions that allow the second binding moiety to bind to a nucleic acid in the labeled biomolecule or labeled gel scaffold of (c), thereby further labeling the biomolecule or gel scaffold with the sequence barcode; and
(e) determining the sequence and location of the nucleic acid.

4. The method of claim 1, wherein the biomolecule is selected from the group consisting of a nucleic acid, a lipid, a small molecule, a sugar, a protein, a nuclei, and a cell.

5. The method of claim 1, wherein the tissue sample is about of 0.1 to 1,000 micron thickness.

6.-8. (canceled)

9. The method of claim 1, wherein preparing the tissue sample optionally comprises contacting the tissue sample or a portion of the tissue sample with a detection agent selected from the group consisting of an antibody, a nucleic acid, a protein, a dye, a bead.

10. The method of claim 9, wherein the tissue sample, prior to preparing, is subjected to a treatment selected from the group consisting of imaging, spectroscopy, mass spectroscopy, enzymatic assays and FISH.

11. The method of claim 10, wherein the detection agent optionally comprises a spatial barcode.

12. (canceled)

13. The method of claim 1, wherein the substrate is a slide, an array, a hydrogel, a tissue, a suspension of particles, or a suspension of droplets.

14. The method of claim 1, wherein the substrate comprises from about 1 to about 4×10¹² first binding moieties.

15. The method of claim 14, wherein the first binding moieties are releasable from the substrate in designated locations.

16. The method of claim 14, wherein the first binding moieties are fixed to the substrate in designated locations.

17. (canceled)

18. The method of claim 1, wherein the spatial barcode molecule is an oligonucleotide.

19.-26. (canceled)

27. The method of claim 1, wherein the second binding moiety comprising the sequence barcode is a bead.

28. The method of claim 27, wherein the bead is a hydrogel bead.

29. The method of claim 1, wherein the second binding moiety comprises a unique sequence barcode associated with from about 1 to about 1×10⁶ primer sequences

30. The method of claim 29, wherein the sequence barcode includes a primer sequence for reverse transcription.

31.-32. (canceled)

33. The method of claim 1, wherein a biomolecule or multiple biomolecules are captured in a hydrogel sample.

34.-39. (canceled)
